(19) United States
(12) Patent Application Publication
(10) Pub. No.: US 2003/0124539 A1
(43) Pub. Date: Jul. 3, 2003

(54) High throughput resequencing and variation detection using high density microarrays

(75) Inventors: Janet A. Warrington, Los Altos, CA (US); Nila A. Shah, Cupertino, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, Inc. A corporation organized under the laws of the State of Delaware

(21) Appl. No.: 10/028,482

(22) Filed: Dec. 21, 2001

(51) Int. Cl. C12Q 1/68; G06F 19/00; G01N 33/48; G01N 33/50; C12M 1/34

(52) U.S. Cl. 435/6; 435/287.2; 702/20

(57) ABSTRACT

In one embodiment of the invention, methods and systems are provided for high thoughput genotyping. The system includes an automated sample preparation system, a sample tracking system, automated array processing and system for data analysis.

### BACKGROUND OF INVENTION

This invention is related to genotyping, laboratory automation, bioinformatics and biological data analysis. Specifically, this invention provides methods, computer software products and systems for analyzing genotyping. Specifically, some embodiments of this invention provide methods, computer software products and systems for comparing nucleotide variant data with gene expression data to obtain a correlation between phenotype and genotype.

Single nucleotide polymorphism (SNP) has been used extensively for genetic analysis. Fast and reliable hybridization-based SNP assays have been developed. (See Wang, et al., Large-Scale Identification, Mapping, and Genotyping of Single-Nucleotide Polymorphism's in the Human Genome, Science 280:1077-1082, 1998; Gingeras, et al., Simultaneous Genotyping and Species Identification Using Hybridization Pattern Recognition Analysis of Generic Mycobacterium DNA Arrays, Genome Research 8:435-448, 1998; Halushka, et al., Patterns of Single-Nucleotide Polymorphisms in Candidate Genes for Blood-Pressure Homeostasis, Nature Genetics 22:239-247, 1999; Cutler, et al., High throughput variation detection and genotyping using microarrays. Genome Research (in press), 2001, all incorporated herein by reference in their entireties.

### SUMMARY OF THE INVENTION

In one aspect of the invention, a system for high throughput detection of genotypes is provided. The exemplary system includes a sample preparation automation system; a sample tracking system; an automated high density probe array loader; a computer system for managing hybridization data and for analyzing hybridization data to make genotype calls.

The sample preparation automation system typically involves a robotic device for handling multiwell plates. In some embodiments, the sample tracking is performed using a machine readable encoding system, for example, a single dimensional or multiple dimensional bar code system or an electromagnetic encoding system.

In some embodiments, the exemplary computer system includes a processor; and a memory being coupled with the processor, the memory storing a plurality of machine instructions that cause the processor to perform the method step of analyzing the hybridization to determine the genotype, where the analyzing comprises calling a genotype by calculating the likelihood of a set of models for the hybridization and the base is called based upon the likelihood of the models; where the distribution of hybridization intensities are assumed to be Gaussein and forward and reverse strand are treated as independent replicates.

The models for the analysis may include five homozygote Models (Null, A, C, G, T) for a haploid and and 11 models (Null, A, C, G, T, A-C, A-G, A-T, C-G, C-T, G-T) for a diploid. In some embodiments, the likelihood of a model is calculated independently for both the forward and reverse strands and is combined for the overall likelihood of the model. A genotype is called if one model fits the hybridization data better than all other models.

In another aspect of the invention, a method for determining the genotype of a polymorphism is provided. In exemplary embodiments, the method includes preparing a nucleic acid sample; determining the hybridization of the nucleic acid sample with a high density oligonucleotide probe array; where the high density oligonucleotide probe array having probes interrogating the polymorphism; and analyzing the hybridization to determine the genotype, where the analyzing comprises calling a genotype by calculating the likelihood of a set of models for the hybridization and the base is called based upon the likelihood of the models.

In some embodiments, the models are five homozygote Models (Null, A, C, G, T) for a haploid and and 11 models (Null, A, C, G, T, A-C, A-G, A-T, C-G, C-T, G-T) for a diploid. The likelihood of a model is calculated independently for both the forward and reverse strands and is combined for the overall likelihood of the model. A genotype is called if one model fits the hybridization data better than all other models. The likelihood of a set of hybridization intensity as measured by pixel intensities is:

$$ln ( L ) = - \frac{1}{2} \sum {N}_{x} [ ln ( {\hat{σ}}_{x}^{2} ) + ( {V}_{x} + {M}_{x}^{2} - 2 {\hat{μ}}_{x} {M}_{x} + {\hat{μ}}_{x}^{2} ) / {\hat{σ}}_{x}^{2} + ln ( 2 π ) ] ,$$

where Nₓ is the number of pixels observed in feature x; Vₓ is the observed variance for feature x, Mₓ is the observed mean for feature x, μₓ is the estimated mean for feature x under a model, and σ²ₓ is the estimated variance for feature x, and wherein the sum is taken over all features x, where x is either A, C, G, or T, on the forward and reverse strands.

The mean and variance for a Null Model are estimated according to:

$${\hat{μ}}_{r} ( b ) = \frac{{N}_{r} ( A ) {M}_{r} ( A ) + {N}_{r} ( C ) {M}_{r} ( C ) + {N}_{r} ( G ) {M}_{r} ( G ) + {N}_{r} ( T ) {M}_{r} ( T )}{{N}_{r} ( A ) + {N}_{r} ( C ) + {N}_{r} ( G ) + {N}_{r} ( T )}$$

$${\hat{μ}}_{f} ( b ) = \frac{{N}_{f} ( A ) {M}_{f} ( A ) + {N}_{f} ( C ) {M}_{f} ( C ) + {N}_{f} ( G ) {M}_{f} ( G ) + {N}_{f} ( T ) {M}_{f} ( T )}{{N}_{f} ( A ) + {N}_{f} ( C ) + {N}_{f} ( G ) + {N}_{f} ( T )}$$

$${\hat{σ}}_{f}^{2} ( b ) = \frac{\begin{matrix}{N}_{f} ( A ) ( {V}_{f} ( A ) + {M}_{f}^{2} ( A ) ) + {N}_{f} ( C ) ( {V}_{f} ( C ) + {M}_{f}^{2} ( C ) ) + \\ {N}_{f} ( G ) ( {V}_{f} ( G ) + {M}_{f}^{2} ( G ) ) + {N}_{f} ( T ) ( {V}_{f} ( T ) + {M}_{f}^{2} ( T ) )\end{matrix}}{{N}_{f} ( A ) + {N}_{f} ( C ) + {N}_{f} ( G ) + {N}_{f} ( T )} - {\hat{μ}}_{f}^{2} ( b )$$

$${\hat{σ}}_{r}^{2} ( b ) = \frac{\begin{matrix}{N}_{r} ( A ) ( {V}_{r} ( A ) + {M}_{r}^{2} ( A ) ) + {N}_{r} ( C ) ( {V}_{r} ( C ) + {M}_{r}^{2} ( C ) ) + \\ {N}_{r} ( G ) ( {V}_{r} ( G ) + {M}_{r}^{2} ( G ) ) + {N}_{r} ( T ) ( {V}_{r} ( T ) + {M}_{r}^{2} ( T ) )\end{matrix}}{{N}_{r} ( A ) + {N}_{r} ( C ) + {N}_{r} ( G ) + {N}_{r} ( T )} - {\hat{μ}}_{r}^{2} ( b )$$

The mean and variance for a hymozygous Model are estimated according to:

$${\hat{μ}}_{f} ( b ) = \frac{{N}_{f} ( C ) {M}_{f} ( C ) + {N}_{f} ( G ) {M}_{f} ( G ) + {N}_{f} ( T ) {M}_{f} ( T )}{{N}_{f} ( C ) + {N}_{f} ( G ) + {N}_{f} ( T )}$$

$${\hat{σ}}_{f}^{2} ( b ) = \frac{{N}_{f} ( C ) {ω}_{f} ( C ) + {N}_{f} ( G ) {ω}_{f} ( G ) + {N}_{f} ( T ) {ω}_{f} ( T )}{{N}_{f} ( C ) + {N}_{f} ( G ) + {N}_{f} ( T )} , \\ {w}_{f} ( x ) = {V}_{f} ( x ) + {M}_{f}^{2} ( x ) - 2 {M}_{f} ( x ) {\hat{μ}}_{f} ( b ) + {\hat{μ}}_{f}^{2} ( b )$$

$${\hat{μ}}_{f} ( A ) = {M}_{f} ( A ) , \\ {\hat{σ}}_{f}^{2} ( A ) = {V}_{f} ( A ) .$$

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 illustrates an example of a computer system that may be utilized to execute the software of an embodiment of the invention.

FIG. 2 is a system block diagram of the computer system of FIG. 1.

FIG. 3 shows a computer network suitable for use with some embodiments of the invention.

FIG. 4 show an exemplary Microarray based high throughput SNP discovery process.

FIG. 5 shows a high-density custom resequencing array. An enlarged portion of a typical image from a scanned array is shown in the inset. The enlarged images on the right show the identical portion of two arrays hybridized with samples from two different individuals whose sequence varies at the second position.

FIG. 6 shows the GeneChip® aray scanner and a scanner autoloader. The scanner autoloader prototype is a refrigerated unit containing 8 racks of 8 arrays and a robotic arm to load and unload the arrays to and from the scanner.

FIG. 7 shows a high throughput fast wash station.

FIG. 8 shows allele frequency verses confidence.

### DETAILED DESCRIPTION OF THE PREFERRED EMBODIMENTS

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention. All cited references, including patent and non-patent literature, are incorporated herein by reference in their entireties for all purposes.

In preferred embodiments, methods are provided for identifying single nucleotide polymorphisms (SNPs) whose state, i.e. wild type (WT), heterozygous (Het), or homozygous (Hom), segregate with gene expression data such that a particular SNP state will correlate with a change in gene expression. The method preferably uses nucleotide variation information derived from hybridization assays in combination with expression information derived from hybridization assays to obtain or predict a correlation between a particular genotype and a particular phenotype.

Various aspects of the invention will be described using SNPs and probe arrays in exemplary embodiments. However, the methods, software and systems are not limited to analyzing biological relevance of SNPs using array based detection technology. Rather, this invention may be applied to, for example, determining functional association between any genotype (such as haplotype) and phenotype. Genotyping can be performed using any suitable technology.

### High Density Probe Arrays

In preferred embodiments, the methods, computer software and systems of the invention are used for analyzing genotyping and gene expression data generated using high density probe arrays, such as high density nucleic acid probe arrays.

High density nucleic acid probe arrays, also referred to as “DNA Microarrays,” have become a method of choice for monitoring the expression of a large number of genes and for detecting sequence variations, mutations and polymorphism. As used herein, “nucleic acids” may include any polymer or oligomer of nucleosides or nucleotides (polynucleotides or oligonucleotidies), which include pyrimidine and purine bases, preferably cytosine, thymine, and uracil, and adenine and guanine, respectively. (See Albert L. Lehninger, PRINCIPLES OF BIOCHEMISTRY, at 793-800 (Worth Pub. 1982) and L. Stryer, BIOCHEMISTRY, 4^{th} Ed. (March 1995), both incorporated by reference.) “Nucleic acids” may include any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“A target molecule” refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51, which is incorporated herein by reference for all purposes. For example, if transcripts of genes are the interest of an experiment, the target molecules would be the transcripts. Other examples include protein fragments, small molecules, etc. “Target nucleic acid” refers to a nucleic acid (often derived from a biological sample) of interest. Frequently, a target molecule is detected using one or more probes. As used herein, a “probe” is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that these are illustrative embodiments that are not to limit the invention in any way.

In preferred embodiments, probes may be immobilized on substrates to create an array. An “array” may comprise a solid support with peptide or nucleic acid or other molecular probes attached to the support. Arrays typically comprise a plurality of different nucleic acids or peptide probes that are coupled to a surface of a substrate in different, localized areas. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. (See Pirrung et al., U.S. Pat. No. 5,143,854, PCT Application No. WO 90/15070) and Fodor et al., PCT Publication Nos. WO 92/10092 and WO 93/09668, U.S. Pat. Nos. 5,677,195, 5,800,992 and 6,156,501, which disclose methods of forming vast arrays of peptides, oligonucleotides and other molecules using, for example, light-directed synthesis techniques.) (See also Fodor, et al., Science, 251, 767-77 (1991)). These procedures for synthesis of polymer arrays are now referred to as VLSIPS™ procedures.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743 and 6,140,044, all of which are incorporated by reference in their entireties for all purposes.

Microarrays can be used in a variety of ways. A preferred microarray contains nucleic acids and is used to analyze nucleic acid samples. Typically, a nucleic acid sample is prepared from appropriate source and labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine fluorescence intensity distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells. (See GATC™ software specification). The probes in a cell are designed to have the same sequence; i.e., each cell is a probe area. A CEL file contains the statistics of a cell, e.g., the 75th percentile and standard deviation of intensities of pixels in a cell. The 50, 60, 70, 75 or 80th percentile of pixel intensity of a cell is often used as the intensity of the cell.

The Affymetrix® Analysis Data Model (AADM) is the relational database schema Affymetrix uses to store experiment results. It includes tables to support mapping, spotted arrays and expression results. Affymetrix publishes AADM to support open access to experiment information generated and managed by Affymetrix® software so that results may be filtered and mined with any compatible analysis tools. The AADM specification (Affymetrix, Santa Clara, Calif., 2001) is incorporated herein by reference for all purposes. The specification is available at http://www.affymetrix.com/support/aadm/aadm.html, last visited on Sep. 4, 2001.

### Genotyping and Polymorphism Detection Using High Density Probe Arrays

Genotyping involves determining the identity of alleles for a gene, genomic regions or regulatory regions or polymorphic marker possessed by an individual. Genotyping of individuals and populations has many uses. Genetic information about an individual can be used for diagnosing the existence or predisposition to conditions to which genetic factors contribute. Many conditions result not from the influence of a single allele, but involve the contributions of many genes. Therefore, determining the genotype for several genomic regions can be useful for diagnosing complex genetic conditions.

Genotyping of many loci from a single individual also can be used in forensic applications, for example, to identify an individual based on biological samples from the individual. Genotyping of populations is useful in population genetics. For example, the tracking of frequencies of various alleles in a population can provide important information about the history of a population or its genetic transformation over time. For a general review of genotyping and its use. (See Diagnostic Molecular Pathology: A Practical Approach: Cell and Tissue Genotyping (Practical Approach Series) by James O'Donnell McGee (Editor), C. S. Herrington (Editor), ISBN: 0199632383 and SNP and Microsatellite Genotyping: Markers for Genetic Analysis (Biotechniques Molecular Laboratory Methods Series.) by Ali Hajeer (Editor), Jane Worthington (Editor), Sally John (Editor), ISBN 1881299384, both are incorporated herein by reference in their entireties.)

Determining the genotype of a sample of genomic material may be carried out using arrays of oligonucleotide probes. These arrays may generally be “tiled” for a contiguous sequence or a large number of specific polymorphisms. In the case of “tiling” for a contiguous sequence, previously unknown sequence variations can be discovered and characterized.

“Tiling,” as used herein, refers to the synthesis of a defined set of oligonucleotide probes which is made up of a sequence complementary to the target sequence of interest, as well as preselected variations of that sequence, e.g., substitution of one or more given positions with one or more members of the basis set of monomers, i.e., nucleotides. Tiling strategies are discussed in detail in, for example, Published PCT Application No. WO 95/11995, incorporated herein by reference in its entirety for all purposes.

One of skill in the art would appreciate that the methods, software and systems of the invention are not limited to any particular tiling format.

Systems for Genotyping Data Analysis

One of skill in the art would appreciate that many computer systems are suitable for carrying out the methods of the invention. Computer software according to the embodiments of the invention can be executed in a wide variety of computer systems.

For a description of basic computer systems and computer networks. (See Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both are incorporated herein by reference in their entireties for all purposes.

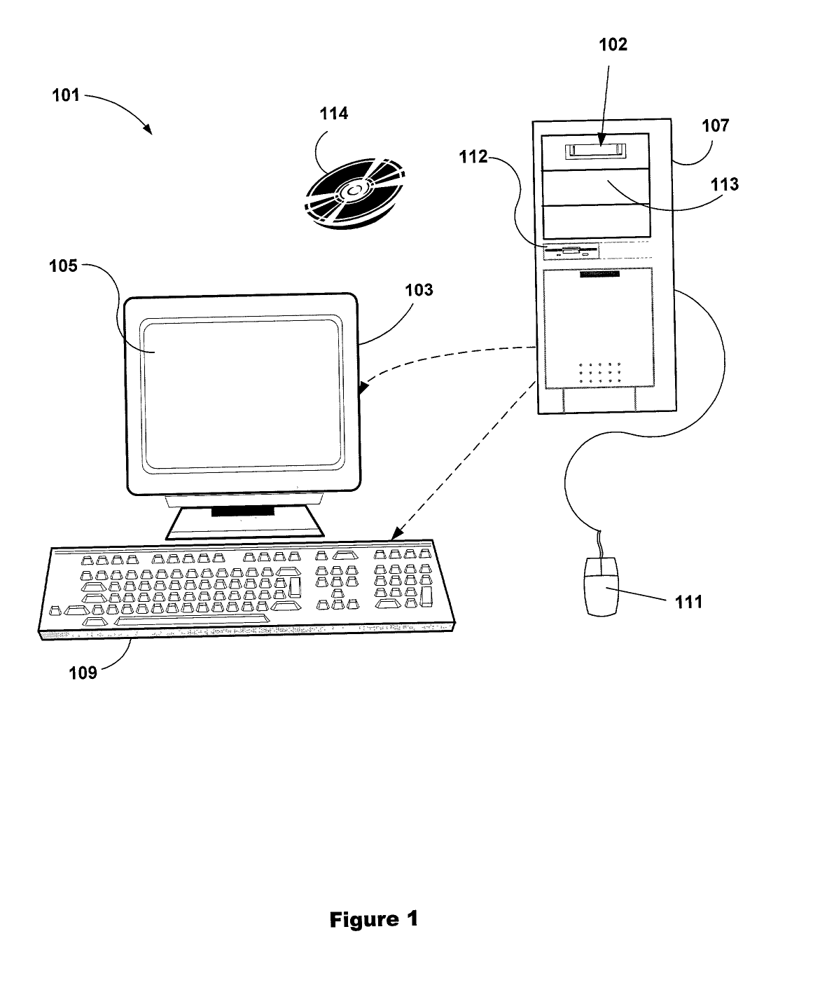

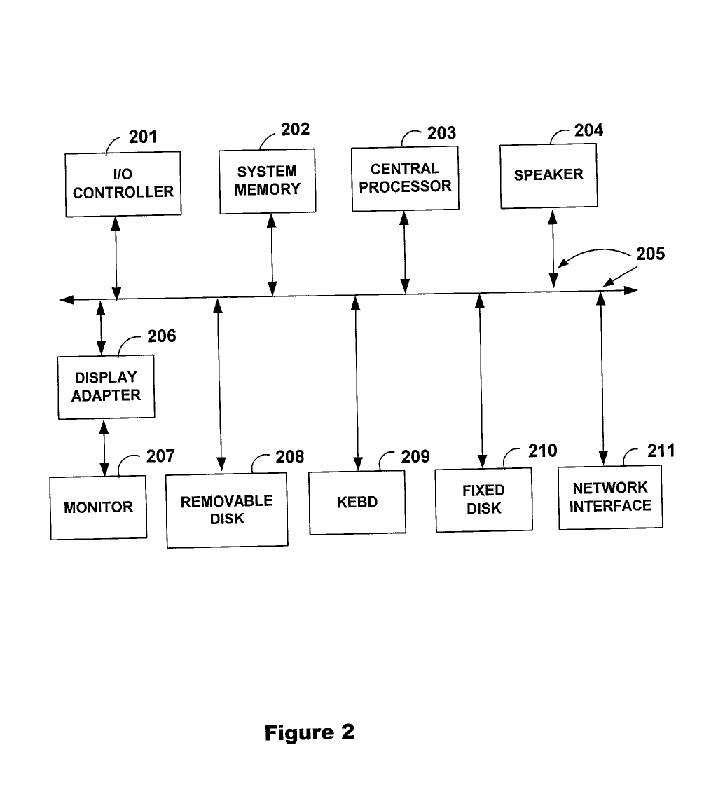

FIG. 1 illustrates an example of a computer system that may be used to execute the software of an embodiment of the invention. FIG. 1 shows a computer system 101 that includes a display 103, screen 105, cabinet 107, keyboard 109, and mouse 111. Mouse 111 may have one or more buttons for interacting with a graphic user interface. Cabinet 107 houses a floppy drive 112, CD-ROM or DVD-ROM drive 102, system memory and a hard drive (113) (see also FIG. 2) which may be utilized to store and retrieve software programs incorporating computer code that implements the invention, data for use with the invention and the like. Although a CD 114 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

FIG. 2 shows a system block diagram of computer system 101 used to execute the software of an embodiment of the invention. As in FIG. 1, computer system 101 includes monitor 201, and keyboard 209. Computer system 101 further includes subsystems such as a central processor 203 (such as a Pentium™ III processor from Intel), system memory 202, fixed storage 210 (e.g., hard drive), removable storage 208 (e.g., floppy or CD-ROM), display adapter 206, speakers 204, and network interface 211. Other computer systems suitable for use with the invention may include additional or fewer subsystems. For example, another computer system may include more than one processor 203 or a cache memory. Computer systems suitable for use with the invention may also be embedded in a measurement instrument.

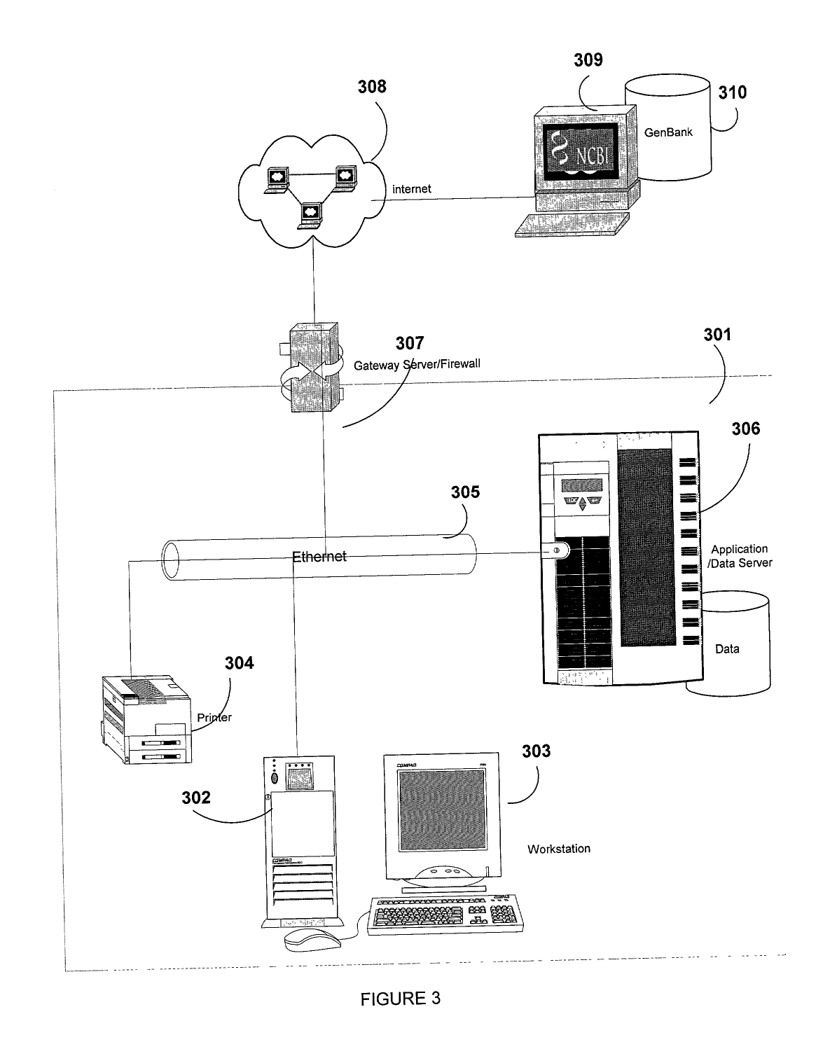

FIG. 3 shows an exemplary computer network that is suitable for executing the computer software of the invention. A computer workstation 302 is connected with and controls a probe array scanner 301. Probe intensities are acquired from the scanner and may be displayed in a monitor 303. The intensities may be processed to make genotype calls (i.e., determining the genotype based upon probe intensities) on the workstation 302. The intensities may be processed and stored in the workstation or in a data server 306. The workstation may be connected with the data server through a local area network (LAN), such as an Ethernet 305. A printer 304 may be connected directly to the workstation or to the Ethernet 305. The LAN may be connected to a wide area network (WAN), such as the Internet 308, via a gateway server 307 which may also serve as a firewall between the WAN 308 and the LAN 305. In preferred embodiments, the workstation may communicate with outside data sources, such as the National Biotechnology Information Center, through the Internet. Various protocols, such as FTP and HTTP, may be used for data communication between the workstation and the outside data sources. Outside genetic data sources, such as the GenBank 310, are well known to those skilled in the art. An overview of GenBank and the National Center for Biotechnology information (NCBI) can be found in the web site of NCBI (http://www.ncbi.nlm.nih.gov).

High Throughput Genotyping Systems

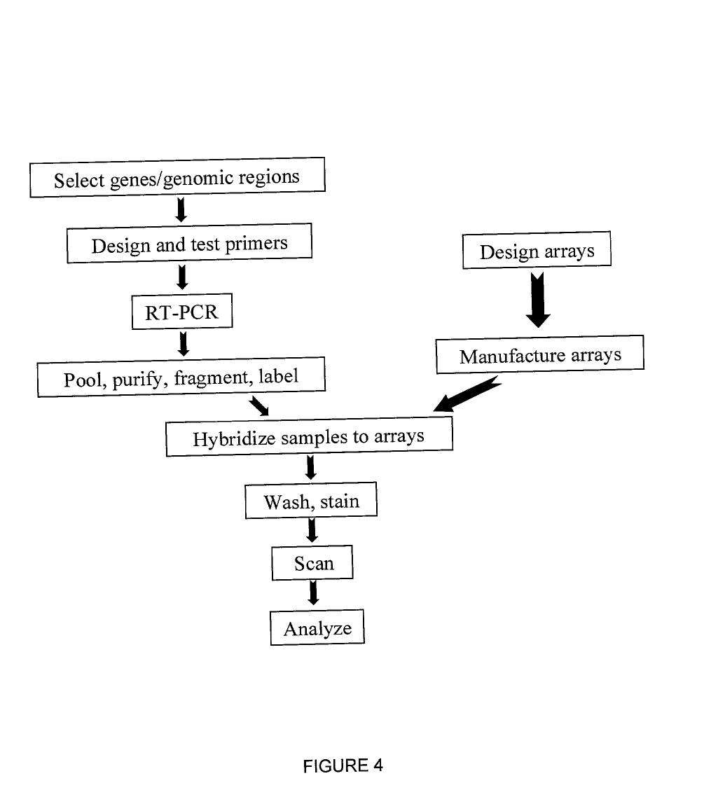

FIG. 4 shows an embodiments of the process for high throughput genotying. Genes or genomic regions are selected. Primers are designed and tested. The validated primers are used to perform RT-PCR or long range PCR. The samples are hybridized with high density oligonucleotide probe arrays.

In one aspect of the invention, a system for high throughput detection of genotypes is provided. The exemplary system includes a sample preparation automation system; a sample tracking system; an automated high density probe array loader; a computer system for managing hybridization data and for analyzing hybridization data to make genotype calls.

The sample preparation automation system typically involves a robotic device for handling multwell plates such as 96 well microtiter plates. In some embodiments, the sample tracking is performed using a machine readable encoding system, for example, a single dimensional or multiple dimensional bar code system or an electromagnetic encoding system. Suitable autoloaders are also described in, for example, U.S. patent application Ser. No. 09/691,702, which is incorporated herein by reference.

In some embodiments, the exemplary computer system includes a processor; and a memory being coupled with the processor, the memory storing a plurality of machine instructions that cause the processor to perform the method step of analyzing the hybridization to determine the genotype.

In some embodiments, the ABACUS system is used to make genotype calls. ABACUS is an Automated Statistical System for Calling VDA Genotypes developed using data generated from Affymetrix Variation Detection Arrays (VDAs).

ABACUS is an automated statistical system for determining individual VDA genotypes whether the site is polymorphic or not. It can be applied in experiments in which the target DNA sequences are either haploid or diploid. In effect, the ABACUS system allows an investigator using VDAs to determine the DNA sequence in a sample of interest. ABACUS has been implemented in ANSI standard C code.

One assumption underlying the ABACUS algorithm is that the observed florescence intensities are normally distributed within features. This assumption is made relying on the central limit theorem. Each feature consists of ˜1 million distinct oligonucleotides of identical composition. If an appreciable fraction of these oligonucleotides are relatively independent in their chance of binding a labeled target, the overall florescence intensity of this feature ought to be normally distributed under some strong version of the central limit theorem. A series of statistical models are developed under the assumption of the presence or absence of various genotypes in the target sample. The likelihood of each statistical model for a given genotype is calculated independently for both the forward and reverse strands and is combined for the overall likelihood of the model. A “quality score,” which is the difference between the log (base 10) likelihood of the best fitting model and the second best fitting model, is assigned to each VDA genotype. A site genotype is “called” when one model fits the data sufficiently better than all other models. After all the individual VDA genotypes are called, additional heuristic, reliability rules are applied. On the completion of this procedure, all sites are assigned a genotype with a corresponding quality score. Individual VDA genotypes deemed unreliable are designated N. The system is divided into six stages.

Stage One: Data Integrity Check.

No Signal. If in a given sample, any feature within any site (either forward or reverse strand) has a mean intensity within two standard deviations of zero, the site is said to have failed in that individual, and this site is ruled N in that individual.

Extremely Weak Signal. If, in a given sample, the highest mean intensity feature on the forward or reverse strand is 20-fold lower than the average highest mean intensity feature, averaged over all samples on that same strand, than this site is said to have failed in this individual, and this genotype is called N in the individual. When this situation occurs at any site, it often occurs over a large number of adjacent sites in the same individual, indicating weak PCR products, improper digestion of sample DNA before hybridization.

Saturation. Among the four features on either the forward or reverse strands, if two (for haploid data) or three (for diploid data) of the features are within two standard deviations of 43,000, the detector is said to have saturated and this site is called N in the given individual. Decreasing the amount of labeled target DNA hybridized to the VDA easily solves saturation.

Aberrant Signal-to-Noise Ratio. The ratio of the mean intensity to the standard deviation of the intensity for a feature will be called the signal-to-noise ratio (SN) of that feature. Over the 57 autosomal VDA designs (˜513 million features), >90% of all features had an SN <20 with a median of ˜8. The tail of the distribution is extremely long, including >100,000 features with an SN above 1000. Sites with one or more features having aberrantly large SN generate aberrantly large likelihoods because as the signal approaches detector limits, it becomes truncated by the detector and appears to have an unusually small variance. As a consequence of these unusually low variances, genotype calls at these sites tend to be highly unreliable. Therefore, to avoid statistical aberrations associated with this, any site with an SN >20 is assigned a variance, so that SN=20.

Stage Two: Building Models With an Even Background

Within any given feature, the florescence intensities of all pixels are assumed to be independent and identically distributed. The distribution is assumed to be Gaussian (normal); forward and reverse strands are treated as independent replicates (with different parameters). The final likelihood for a model is calculated by multiplying the likelihood on the forward strand by the likelihood on the reverse strand. Therefore, the log (base e) likelihood of a set of pixel florescence intensities is given by:

$$ln ( L ) = - \frac{1}{2} \sum {N}_{x} [ ln ( {\hat{σ}}_{x}^{2} ) + ( {V}_{x} + {M}_{x}^{2} - 2 {\hat{μ}}_{x} {M}_{x} + {\hat{μ}}_{x}^{2} ) / {\hat{σ}}_{x}^{2} + ln ( 2 π ) ] ,$$

where Nₓ is the number of pixels observed in feature x (Nₓ generally is equal to 30, but this number can vary slightly with imperfect grid alignment), Vₓ is the observed variance for feature x, Mₓ is the observed mean for feature x, μₓ is the estimated mean for feature x under the model in question, and σ²ₓ is the estimated variance for feature x. The sum is taken over all features x, where x is either A, C, G, or T, on the forward and reverse strands.

Null Model

All features on the forward strand are assumed to have identical means and variances. All features on the reverse strand are assumed to have identical means and variances, but these may differ between the two strands; these parameters are set equal to their maximum likelihood estimators. Maximum likelihood estimates can be found by differentiating Equation 1, with respect to all parameters and solving simultaneously. This results in the naive estimators, which are

$${\hat{μ}}_{f} ( b ) = \frac{{N}_{f} ( A ) {M}_{f} ( A ) + {N}_{f} ( C ) {M}_{f} ( C ) + {N}_{f} ( G ) {M}_{f} ( G ) + {N}_{f} ( T ) {M}_{f} ( T )}{{N}_{f} ( A ) + {N}_{f} ( C ) + {N}_{f} ( G ) + {N}_{f} ( T )}$$

$${\hat{μ}}_{r} ( b ) = \frac{{N}_{r} ( A ) {M}_{r} ( A ) + {N}_{r} ( C ) {M}_{r} ( C ) + {N}_{r} ( G ) {M}_{r} ( G ) + {N}_{r} ( T ) {M}_{r} ( T )}{{N}_{r} ( A ) + {N}_{r} ( C ) + {N}_{r} ( G ) + {N}_{r} ( T )}$$

$${\hat{σ}}_{f}^{2} ( b ) = \frac{\begin{matrix}{N}_{f} ( A ) ( {V}_{f} ( A ) + {M}_{f}^{2} ( A ) ) + {N}_{f} ( C ) ( {V}_{f} ( C ) + {M}_{f}^{2} ( C ) ) + \\ {N}_{f} ( G ) ( {V}_{f} ( G ) + {M}_{f}^{2} ( G ) ) + {N}_{f} ( T ) ( {V}_{f} ( T ) + {M}_{f}^{2} ( T ) )\end{matrix}}{{N}_{f} ( A ) + {N}_{f} ( C ) + {N}_{f} ( G ) + {N}_{f} ( T )} - {\hat{μ}}_{f}^{2} ( b )$$

$${\hat{σ}}_{r}^{2} ( b ) = \frac{\begin{matrix}{N}_{r} ( A ) ( {V}_{r} ( A ) + {M}_{r}^{2} ( A ) ) + {N}_{r} ( C ) ( {V}_{r} ( C ) + {M}_{r}^{2} ( C ) ) + \\ {N}_{r} ( G ) ( {V}_{r} ( G ) + {M}_{r}^{2} ( G ) ) + {N}_{r} ( T ) ( {V}_{r} ( T ) + {M}_{r}^{2} ( T ) )\end{matrix}}{{N}_{r} ( A ) + {N}_{r} ( C ) + {N}_{r} ( G ) + {N}_{r} ( T )} - {\hat{μ}}_{r}^{2} ( b )$$

where \hat{μ}_{f}(b) and \hat{μ}ᵣ(b) are the estimated mean background intensities on the forward and reverse strands, respectively. The \hat{σ}²s are the analogous variances. Let L_{f}(0) and Lᵣ(0) be the likelihoods of the null model restricted to the forward or reverse strand, respectively. L(0)=L_{f}(0)Lᵣ(0) is the overall likelihood of the null model.

Homozygote Models

Consider the hypothesis that the sample is an A homozygote. Under this model, features C, G, and T on the forward strand are assumed to be independent and identically distributed. The background mean and background variance is estimated by maximum likelihood to be

$${\hat{μ}}_{f} ( b ) = \frac{{N}_{f} ( C ) {M}_{f} ( C ) + {N}_{f} ( G ) {M}_{f} ( G ) + {N}_{f} ( T ) {M}_{f} ( T )}{{N}_{f} ( C ) + {N}_{f} ( G ) + {N}_{f} ( T )}$$

$${\hat{σ}}_{f}^{2} ( b ) = \frac{{N}_{f} ( C ) {w}_{f} ( C ) + {N}_{f} ( G ) {w}_{f} ( G ) + {N}_{f} ( T ) {w}_{f} ( T )}{{N}_{f} ( C ) + {N}_{f} ( G ) + {N}_{f} ( T )} , \\ {w}_{f} ( x ) = {V}_{f} ( x ) + {M}_{f}^{2} ( x ) - 2 {M}_{f} ( x ) {\hat{μ}}_{f} ( b ) + {\hat{μ}}_{f} ( b ) + {\hat{μ}}_{f}^{2} ( b )$$

Feature A on the forward strand is assumed to have a different mean and variance, and these are estimated by maximum likelihood to be the observed values. Therefore,

\hat{μ}_{f}(A)=M_{f}(A)

\hat{σ}²_{f}(A)=V_{f}(A)

The reverse strand is treated analogously.

Let Lf(A) and Lr(A) be the likelihoods of the A homozygote model restricted to the forward strand and reverse strand, respectively. If the estimated mean for A is less than the estimated mean for the background, the likelihood is set equal to the null model likelihood. Therefore, if f(A)<f(b) then Lf(A)=Lf(0). Similarly, if r(T)<r(b) then Lr(A)=Lr(0). L(A) is the overall likelihood of the A homozygote model, L(A)=Lf(A)Lr(A). All other homozygote models are treated analogously.

Heterozygote Models

When examining diploid data, six (A-C, A-G, A-T, C-G, C-T, G-T) heterozygote models, beyond the four homozygote models, are also considered. Consider an A-C heterozygote. Background features G and T on the forward strand are assumed to be independent and identically distributed. The mean and variance is estimated by maximum likelihood (See below). Features A and C on the forward strand are assumed to be independent and identically distributed, and parameter estimates are given below.

Stage 3: Compare Models

For haploid data, a total of five models are examined (Null, A, C, G, T). For diploid data, a total of 11 models are examined (Null, A, C, G, T, AC, AG, AT, CG, CT, GT).

Quality Scores for Each Model

For each model, three quality scores are calculated. For Model A, Qf(A)=Log10(Lf(A)) Log10(Lf(max other)), where Lf(max other) is the maximum over all models other than A (also notice that these logs are taken base 10, not base e). Therefore, Qf(A) is the difference between the log likelihood of model A on the forward strand and the best fitting model on the forward strand, excluding A. If Qf(A) is positive, A is the best fitting model on the forward strand. Qf(A) will be called as the quality score for model A on the forward strand. Qr(A)=Log10(Lr(A)) Log10(Lr(max_other)) is the analogous quality score on the reverse strand. The overall quality score for model A is Q(A)=Log10(L(A)) Log10(L(max_other)). Therefore, Q(A) is the difference between the likelihood of model A, overall, and the best fitting model, excluding A, overall. If Q(A) is positive, A is the best fitting model, overall. Similar statistics are calculated for all other models.

In addition, two further likelihoods may be calculated: Lf(Perfect) and Lr(Perfect). These likelihoods correspond to the likelihood of the best possible fitting model on the forward and the best possible fitting model on the reverse strand. A “perfect” fitting model is defined by the predicted mean intensity for all features equaling the observed mean, and the predicted variance for all features equaling the observed variance. This “perfect fit” model is simply the unconstrained, fully parameterized model. All other models are nested within it. Therefore, Lf(Perfect) is the largest likelihood possible on the forward strand, and Lr(Perfect) is the largest likelihood possible on the reverse strand.

There are two set of criteria (quality thresholds) used to call a site. One set of quality thresholds corresponds to a single model fitting the data exceptionally well (nearly perfectly). A second, more stringent set of requirements, corresponds to no model fitting nearly perfectly, but one model fitting the data much better than any other model.

Calling a Near-Perfect Fit

The perfect fitting model has eight parameters per strand. Any particular genotype model has four parameters per strand, and each of these models is nested within the perfect fitting model. Therefore, standard likelihood ratio tests can be used to compare the fit of any particular model with the perfect fitting model. Therefore, Df=2[ln(Lf(perfect)) ln(Lf(model))] ought to be 2 distributed with 4 degrees of freedom (Dr is defined similarly). A model is to fit nearly perfectly if Df and Dr are sufficiently small. Sufficiently small may be defined as <6.63 (˜85% confidence interval).

When one model fits nearly perfectly, and all other models fit much more poorly, we will call this model a near-perfect fit. Comparing the fit of one model to another is not straight forward, as these models are not nested and have the same number of parameters. If it is assumed that the difference in the fit between any two non-nested models is 2 distributed with 1 degree of freedom, then Qf(near-perfect fit model) >5.2 would imply that there is less than a 106 chance that the difference in fit is attributable to chance. Therefore, if any model fits nearly perfectly, with Qf(model)>5.2 and Qr(model)>5.2, then the genotype associated with this model is called.

Calling an Imperfect Fit

It is rare for any model to fit nearly perfectly. When no model fits nearly perfectly, there is no obvious way to relate quality scores to statistical probabilities. With no a priori predictions for what a good quality score ought to be, quality scores necessary to call a model have been determined empirically by examining the data generated from this project. Two thresholds for quality scores have been established, a “total threshold,” Ttotal, and a “strand threshold,” Tstrand. A model is said to fit significantly better than any other model when Q(model)>Ttotal, and Qf(model)>Tstrand and Qr(model) >Tstrand. When one model fits significantly better than all others, the genotype associated with this model is called. For the experiments described in this paper, Ttotal has been chosen to be 30, and Tstrand has been chosen to be 2 (justification is described below).

If, for a given sample, no model can be called either a near-perfect fit, or an imperfect fit, N is assigned to this genotype.

Stage 4: Building Models With an Uneven Background (For Diploid Data Only)

All of the previous modeling (Stages 2 and 3) assumed that all background features had identical means and variances. Moreover, background features with uneven means can appear very similar to heterozygotes. The uneven background models assume that the background features have means and variances that are constant ratios of each other. These ratio constants (s and s in the notation below) are obtained by averaging over all samples with the same genotype. The genotypes and the background can be inferred in an iterative manner, changing the background constants as genotype calls change.

The following section also summerizes the models behind Abacus: Even Background.

Heterozygote Models—When examining diploid data, six {A-C, A-G, A-T, C-G, C-T, G-T} heterozygote models, beyond the four homozygote models, are also considered. Consider an A-C heterozygote. Background features G and T on the forward strand are assumed to be independent and identically distributed. The mean and variance is estimated by maximum likelihood to be

$${\hat{u}}_{f} ( b ) = \frac{{N}_{f} ( G ) {M}_{f} ( G ) + {N}_{f} ( T ) {M}_{f} ( T )}{{N}_{f} ( G ) + {N}_{f} ( T )} . \\ \begin{matrix}{\hat{σ}}_{f}^{2} ( b ) = \frac{{N}_{f} ( G ) {ω}_{f} ( G ) + {N}_{f} ( T ) {ω}_{f} ( T )}{{N}_{f} ( G ) + {N}_{f} ( T )} \\ {ω}_{f} ( x ) = {V}_{f} ( x ) + {M}_{f}^{2} ( x ) - 2 {M}_{f} ( x ) {\hat{μ}}_{f} ( b ) + {\hat{μ}}_{f}^{2} ( b )\end{matrix} .$$

Features A and C on the forward strand are assumed to be independent and identically distributed. The mean and variance is estimated to be the maximum likelihood estimates of mean and variance over all these pixels.

$${\hat{u}}_{f} ( A C ) = \frac{{N}_{f} ( A ) {M}_{f} ( A ) + {N}_{f} ( C ) {M}_{f} ( C )}{{N}_{f} ( A ) + {N}_{f} ( C )} . \\ \begin{matrix}{\hat{σ}}_{f}^{2} ( A C ) = \frac{{N}_{f} ( A ) {ω}_{f} ( A ) + {N}_{f} ( C ) {ω}_{f} ( C )}{{N}_{f} ( A ) + {N}_{f} ( C )} \\ {ω}_{f} ( x ) = {V}_{f} ( x ) + {M}_{f}^{2} ( x ) - 2 {M}_{f} ( x ) {\hat{μ}}_{f} ( A C ) + {\hat{μ}}_{f}^{2} ( A C )\end{matrix} .$$

The reverse strand is treated analogously. Let L_{f}(AC), Lᵣ(AC), and L(AC)=L_{f}(AC)Lᵣ(AC) be the likelihoods of the AC model on the forward strand, reverse strand, and overall, respectively. If μ_{f}(AC)<μ_{f}(b) then L_{f}(AC)=L_{f}(0). Similarly if μᵣ(GT)<μᵣ(b) then Lᵣ(AC)=Lᵣ(0).

All other heterozygote models are treated analogously.

Uneven Background Models

Homozygote A—Feature C on the forward strand is assumed normal with mean μ_{f}(b) and variance σ²_{f}(b). Feature G on the forward strand is assumed normal with mean β_{f}(G/C)μ_{f}(b) and variance α_{f}(G/C)σ²_{f}(b). Feature T on the forward strand is assumed normal with mean β_{f}(T/C)μ_{f}(b) and variance α_{f}(T/C)σ²_{f}(b). All means and all variances are fit by maximum likelihood, assuming that the α's and β's are constants. Thus,

\hat{μ}_{f} (C)=\hat{μ}_{f}(b)

\hat{μ}ᵣ(G)=\hat{μ}ᵣ(b)

\hat{μ}_{f}(G)=β_{f}(G/C)\hat{μ}_{f}(b).

\hat{μ}ᵣ(C)=βᵣ(C/G)\hat{μ}ᵣ(b)

\hat{μ}_{f}(T)=β_{f}(T/C)\hat{μ}_{f}(b)

\hat{μ}ᵣ(A)=βᵣ(A/G)\hat{μ}ᵣ(b)

\hat{σ}_{f}²(C)=\hat{σ}_{f}²(b)

\hat{σ}_{f}²(G)=\hat{σ}_{f}²(b)

\hat{σ}_{f}²(G)=α_{f}(G/C)\hat{σ}_{f}²(b).

\hat{σ}ᵣ²(C)=αᵣ(C/G)\hat{σ}ᵣ²(b)

\hat{σ}_{f}²(T)=α_{f}(T/C)\hat{σ}_{f}²(b)

\hat{σ}ᵣ²(A)=αᵣ(A/G)\hat{σ}ᵣ²(b)

Differentiation of equation 1, and simultaneous solution yields maximum likelihood estimation of means and variances

$${\hat{u}}_{f} ( b ) = \frac{\begin{matrix}{N}_{f} ( C ) {M}_{f} ( C ) {α}_{f} ( G / C ) {a}_{f} ( T / C ) + \\ {N}_{f} ( G ) {M}_{f} ( G ) {β}_{f} ( G / C ) {a}_{f} ( T / C ) + \\ {N}_{f} ( T ) {M}_{f} ( T ) {α}_{f} ( G / C ) {β}_{f} ( T / C )\end{matrix}}{\begin{matrix}\begin{matrix}{N}_{f} ( C ) {α}_{f} ( G / C ) {a}_{f} ( T / C ) + \\ {N}_{f} ( G ) {β}_{f}^{2} ( G / C ) {a}_{f} ( T / C ) +\end{matrix} \\ {N}_{f} ( T ) {α}_{f} ( G / C ) {β}_{f}^{2} ( T / C )\end{matrix}} . \\ {\hat{u}}_{r} ( b ) = \frac{\begin{matrix}{N}_{r} ( G ) {M}_{fr} ( G ) {α}_{r} ( C / G ) {a}_{r} ( A / G ) + \\ {N}_{r} ( C ) {M}_{r} ( C ) {β}_{r} ( C / G ) {a}_{r} ( A / G ) + \\ {N}_{r} ( A ) {M}_{r} ( A ) {α}_{r} ( C / G ) {β}_{r} ( A / G )\end{matrix}}{\begin{matrix}\begin{matrix}{N}_{r} ( G ) {α}_{r} ( C / G ) {a}_{r} ( A / G ) + \\ {N}_{r} ( C ) {β}_{r}^{2} ( C / G ) {a}_{r} ( A / G ) +\end{matrix} \\ {N}_{r} ( A ) {α}_{r} ( C / G ) {β}_{r}^{2} ( A / G )\end{matrix}}$$

$${\hat{σ}}_{f}^{2} ( b ) = \frac{{N}_{f} ( C ) {ω}_{f} ( C ) + {N}_{f} ( G ) {ω}_{f} ( G ) + {N}_{f} ( T ) {ω}_{f} ( T )}{{α}_{f} ( G / C ) {α}_{f} ( T / C ) [ {N}_{f} ( C ) + {N}_{f} ( G ) + {N}_{f} ( T ) ]} , \\ {\hat{σ}}_{r}^{2} ( b ) = \frac{{N}_{r} ( G ) {ω}_{r} ( G ) + {N}_{r} ( C ) {ω}_{r} ( C ) + {N}_{r} ( A ) {ω}_{r} ( A )}{{α}_{r} ( C / G ) {α}_{r} ( A / G ) [ {N}_{r} ( G ) + {N}_{r} ( C ) + {N}_{r} ( A ) ]}$$

where,

ω_{f}(C)=α_{f}(G/C)α_{f}(T/C)[V_{f}(C)+M_{f}²(C)−2M_{f}(C)\hat{μ}_{f}(b)+\hat{μ}_{f}²(b)]

ωᵣ(G)=αᵣ(C/G)αᵣ(A/G)[Vᵣ(C)+Mᵣ²(G)−2Mᵣ(G)\hat{μ}ᵣ(b)+\hat{μ}ᵣ²(b)]

ω_{f}(G)=α_{f}(T/C)[V_{f}(G)+M²_{f}(G)=2M_{f}(G)\hat{μ}_{f}(b)β_{f}(G/C)+\hat{μ}_{f}²(b)β_{f}²(G/C)]

ωᵣ(C)=αᵣ(A/G)[Vᵣ(C)+Mᵣ²(C)−2Mᵣ(C)\hat{μ}ᵣ(b)βᵣ(C/G)+\hat{μ}ᵣ²(b)βᵣ²(C/G)]

ω_{f}(T)=α_{f}(G/C)[V_{f}(T)+M_{f}²(T)−2M_{f}(T)\hat{μ}_{f}(b)β_{f}(T/C)+\hat{μ}_{f}²(b)β_{f}²(T/C)]

ωᵣ(A)=αᵣ(C/G)[Vᵣ(A)+Mᵣ²(A)−2Mᵣ(A)\hat{μ}ᵣ(b)βᵣ(A/G)+\hat{μ}ᵣ²(b)βᵣ²(A/G)]

Determination of the α's and β's is discussed below. Estimated moments for feature A are unaffected by the background, and given by equation 4. All other homozygous features are similarly treated.

Heterozygote A-C—Feature G on the forward strand is assumed normal with mean μ_{f}(b) and variance σ²_{f}(b). Feature T on the forward strand is assumed normal with mean β_{f}(T/G)μ_{f}(b) and variance α_{f}(T/G)σ²_{f}(b). All means and variances are fit by maximum likelihood, under the assumption that the α's and β's are constants. Thus,

\hat{μ}_{f}(G)=\hat{μ}_{f}(b)

\hat{μ}ᵣ(C)=\hat{μ}ᵣ(b)

\hat{μ}_{f}(T)=β_{f}(T/G)\hat{μ}_{f}(b)

\hat{μ}ᵣ(A)=βᵣ(A/C)\hat{μ}ᵣ(b)

\hat{σ}_{f}²(G)=\hat{σ}_{f}²(b)

\hat{σ}ᵣ²(C)=\hat{σ}ᵣ²(b)

\hat{σ}_{f}²(T)=α_{f}(T/G)\hat{σ}_{f}²(b)

\hat{σ}ᵣ²(A/C)\hat{σ}ᵣ²(b)

$${\hat{μ}}_{f} ( b ) = \frac{{N}_{f} ( G ) {α}_{f} ( T / G ) {M}_{f} ( G ) + {N}_{f} ( T ) {β}_{f} ( T / G ) {M}_{f} ( T )}{{N}_{f} ( G ) {α}_{f} ( T / G ) + {N}_{f} ( T ) {β}_{f}^{2} ( T / G )}$$

$${\hat{μ}}_{r} ( b ) = \frac{{N}_{r} ( C ) {α}_{r} ( A / C ) {M}_{r} ( C ) + {N}_{r} ( A ) {β}_{r} ( A / C ) {M}_{r} ( A )}{{N}_{r} ( C ) {α}_{r} ( A / C ) + {N}_{r} ( A ) {β}_{r}^{2} ( A / C )}$$

$${\hat{σ}}_{f}^{2} ( b ) = \frac{\begin{matrix}{N}_{f} ( G ) {α}_{f} ( T / G ) [ {V}_{f} ( G ) + {M}_{f}^{2} ( G ) - 2 {M}_{f} ( G ) {\hat{μ}}_{f} ( b ) + \\ \begin{matrix}{μ}_{f}^{2} ( b ) ] + {N}_{f} ( T ) [ {V}_{f} ( T ) + {M}_{f}^{2} ( T ) - \\ 2 {M}_{f} ( T ) {\hat{μ}}_{f} ( b ) {β}_{f} ( T / G ) + {μ}_{f}^{2} ( b ) {β}_{f}^{2} ( T / G )\end{matrix}\end{matrix}}{{α}_{f} ( T / G ) [ {N}_{f} ( G ) + {N}_{f} ( T ) ]}$$

$${\hat{σ}}_{r}^{2} ( b ) = \frac{\begin{matrix}{N}_{r} ( C ) {α}_{r} ( A / C ) [ {V}_{r} ( C ) + {M}_{r}^{2} ( C ) - 2 {M}_{r} ( C ) {\hat{μ}}_{r} ( b ) + {μ}_{r}^{2} ( b ) ] + \\ {N}_{r} ( A ) [ {V}_{r} ( A ) + {M}_{r}^{2} ( A ) - 2 {M}_{r} ( A ) {\hat{μ}}_{r} ( b ) {β}_{r} ( A / C ) + {μ}_{r}^{2} ( b ) {β}_{r}^{2} ( A / C )\end{matrix}}{{α}_{r} ( A / C ) [ {N}_{r} ( C ) + {N}_{r} ( A ) ]}$$

Features A and C on the forward strand are assumed independent and identically distributed with mean and variance given by equations 6 and 7. All other heterozygote models are treated analogously. As usual, likelihood and Quality Scores are calculated for all ten models.

Determination of the α's and β's.—Consider the A homozygote model for a specific sample. β_{f}(G/C) is estimated to be the mean florescence at feature G, divided by the mean florescence at feature C, where both averages are taken over all samples previously called A. If no sample, other than this sample, has been called A previously, averaging occurs over all samples called or guessed to be A. If no sample, other than this sample, has been called or guessed A, and less than 10% of all samples have been called, the average is taken over all samples designated N. Otherwise, β_{f}(G/C) is set equal to 1.0. α_{f}(G/C) is the corresponding quantity averaged over observed variances. All other constants are treated analogously. Thus,

$$\begin{matrix}{α}_{f} ( G / C ) = \frac{\sum [ {N}_{f} ( G ) {V}_{f} ( G ) ] \sum {N}_{f} ( C )}{\sum [ {N}_{f} ( C ) {V}_{f} ( C ) ] \sum {N}_{f} ( G )} \\ {α}_{f} ( T / C ) = \frac{\sum [ {N}_{f} ( T ) {V}_{f} ( T ) ] \sum {N}_{f} ( C )}{\sum [ {N}_{f} ( C ) {V}_{f} ( C ) ] \sum {N}_{f} ( T )} \\ {β}_{f} ( G / C ) = \frac{\sum [ {N}_{f} ( G ) {M}_{f} ( G ) ] \sum {N}_{f} ( C )}{\sum [ {N}_{f} ( C ) {M}_{f} ( C ) ] \sum {N}_{f} ( G )} \\ {β}_{f} ( T / C ) = \frac{\sum [ {N}_{f} ( T ) {M}_{f} ( T ) ] \sum {N}_{f} ( C )}{\sum [ {N}_{f} ( C ) {M}_{f} ( C ) ] \sum {N}_{f} ( T )}\end{matrix}$$

### EXAMPLE

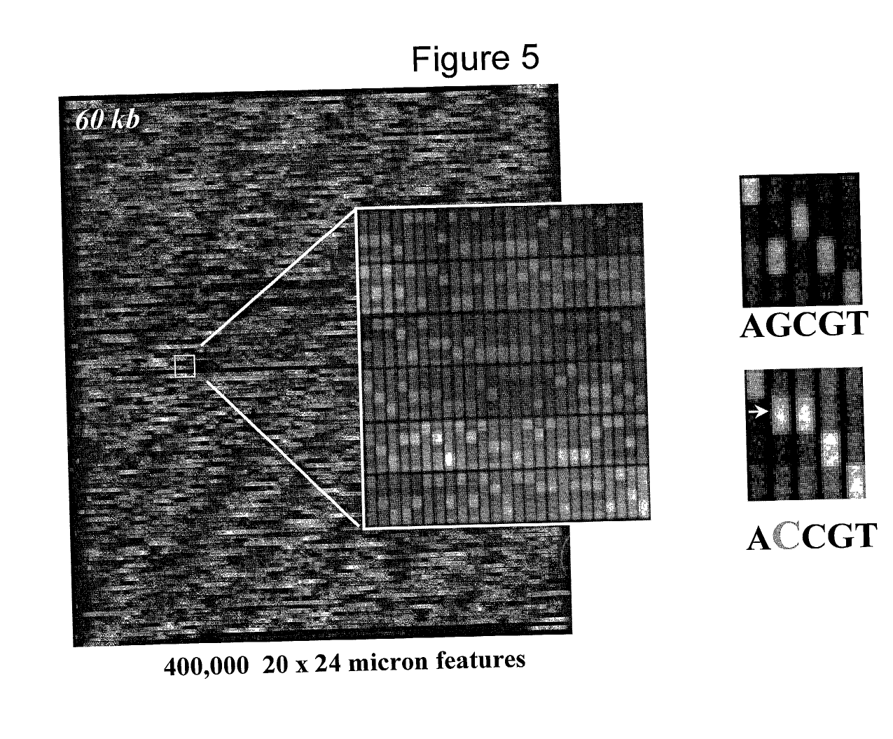

This section describes a high throughput system for resequencing for SNP discovery using high density microarrays. This example illustrates various aspects of the invention. A number of improvements in sample preparation methods, hybridization assay, array handling and analysis method were developed and implemented. DNA from forty unrelated individuals of three different ethnic origins was amplified, labeled and hybridized to arrays designed with probes representing genomic, coding and regulatory regions. Protocol improvements including the use of long PCR and semi-automation reduced labeling and fragmentation costs by 33%. Automation improvements include the development of a scanner autoloader for arrays, a faster array wash station, and a linked laboratory tracking and data management system. Validation of a smaller feature size, 20×24 microns, allowed the simultaneous screening of 30 kb sense and 30 kb antisense DNA (FIG. 5) on each microarray, increasing throughput to 1.4 Mb per day per two laboratory personnel. More than 15,000 SNPs were identified in 8.3 MB of the human genome using high-density resequencing and variation detection arrays (microarray).

Generally the goal of the project was to reduce the cost of array based resequencing by implementing changes in every aspect of the protocol. Specifically, increasing the amount of information obtained per array by reducing the feature size and validating the quality of the data obtained from reduced signal, develop an improved. Less costly sample preparation method, most significantly reduce the PCR primer cost and sample volumes, automate sample preparation and chip handling at the bench, add internal controls for monitoring array performance, develop an improved base-calling algorithm, improve base calling and SNP calling accuracy. Advancements were made incrementally and as throughput increased and the cost of SNP discovery dropped, data quality improved (Cargill M, Altshuler D, Ireland J, Sklar P, Ardlie K, Patil N, Shah N A, Lane C R, Lim E, Kalyanaraman N, Nemesh J, Ziaugra L, Friedland L, Rolfe A, Warrington J A, Lipshutz R, Daley G Q, Lander E S. 1999. Characterization of single-nucleotide polymorphisms in coding regions of human genes. Nat Genet 22: 231-238; indblad-Toh K, Winchester E, Daly M J, Wang D G, Hirschhorn J N, Laviolette J-P, Ardlie K, Reich D E, Robinson E, Sklar P, Shah N, Thomas D, Fan J-B, Gingeras T, Warrington J, Patil N, Hudson T J, Lander E S. 2000. Large-scale discovery and genotyping of single nucleotide polymorphisms in the mouse. Nature Genet 24: 381-386; 1. Cutler D J, Zwick M E, Carrasquillo M M, Yohn C T, Tobin K P, Kashuk C, Mathews D J, Shah N A, Eichler E E, Warrington J A, Chakravarti A. 2001. High throughput variation detection and genotyping using microarrays. Genome Res 11(11):1913-25).

Materials and Methods

Sample source. Cell lines from the NIH Coriell diversity panel were used as a source of genomic DNA or mRNA, for preparation of cDNA (Coriell Institute, Camden, N.J.). Samples were selected to represent 40 males and females of three different ethnic origins, Northern European, 11 females and 9 males, African, 10 females, and Asian, 4 females and 6 males.

Primer design. After genes or genomic regions of interest were identified, PCR primers were designed in preparation for carrying out long PCR to produce amplicons ranging from 3-15 KB, using a variety of publicly and commercially available programs, i.e., Primer 3 (www-genome.wi.mit.edu/cgi-bin/primer/primer3_www.cgi), Amplify 1.2 (Engels et al. 1993), Oligo 6 (SR Lifescience,www.lifescience-software.com). Primers were tested on a pool of DNA produced from three different Coriell samples, cDNA or genomic DNA depending on the project.

Sample preparation. Genomic DNA was isolated using standard methods (Moore et al., 1984). cDNA was prepared from mRNA as previously described (Mahadevappa M, Warrington J A. 1999. A high-density probe array sample preparation method using 10-100-fold fewer cells. Nat. Biotechnol 17:1134-1136). Samples were amplified using long PCR of the region of interest and an aliquot of each amplicon was electrophoresed to confirm size and quantity prior to pooling as previously described (Cutler et al. 2001). A Multiplex II model MPIIEX robot was used for setting up PCR reactions, amplicon pooling, concentration and purification steps (Packard Instrument Co., Meriden, Conn.). Expression analysis. To optimize PCR success when cDNA was being used as the PCR template, expression analysis was carried out to determine the relative abundance of each transcript and to identify unexpressed genes and transcripts of interest that may be too low in abundance to amplify robustly from the lymphoblast cell lines. Expression analysis was carried out on an array containing probes representing 6800 fall length human genes, HuGeneFL® probe array (Affymetrix Inc., Santa Clara, Calif.). The samples were prepared and the arrays hybridized following manufacturer instructions (Affymetrix Inc., Santa Clara, Calif.). Copy numbers are determined by correlating the known concentrations of the spiked standards with their hybridization intensities as previously described (Lockhart et al. 1996). Transcript abundance is calculated assuming an average of 300,000 transcripts per cell with an average transcript size of 1 kb.

Custom Resequencing Arrays

High-density resequencing or variation detection arrays, i.e. SNP discovery arrays, were designed to correspond with DNA fragments successfully amplified by long PCR. Each array contains 0.5 KB of actin sequence to be used as an internal laboratory control as well as a set of standard controls that were used for quality control in manufacturing. Each custom design contains ˜400,000 different probes representing 30 KB of sense and 30 KB of antisense DNA (FIG. 2). Each of the 400,000 different probes resides in a 20 micron×24 micron feature and each feature contains millions of identical copies of the same probe.

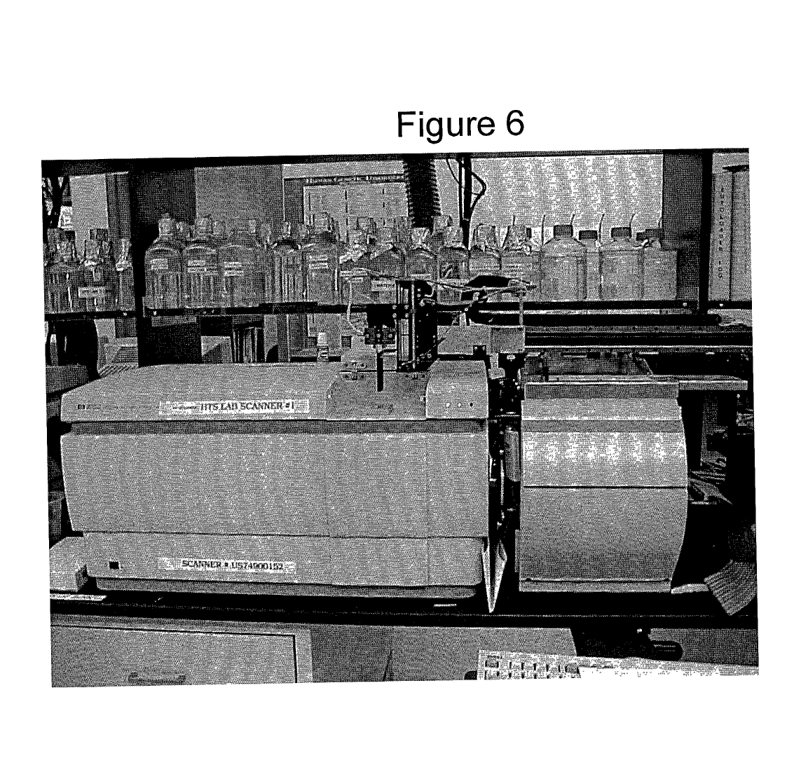

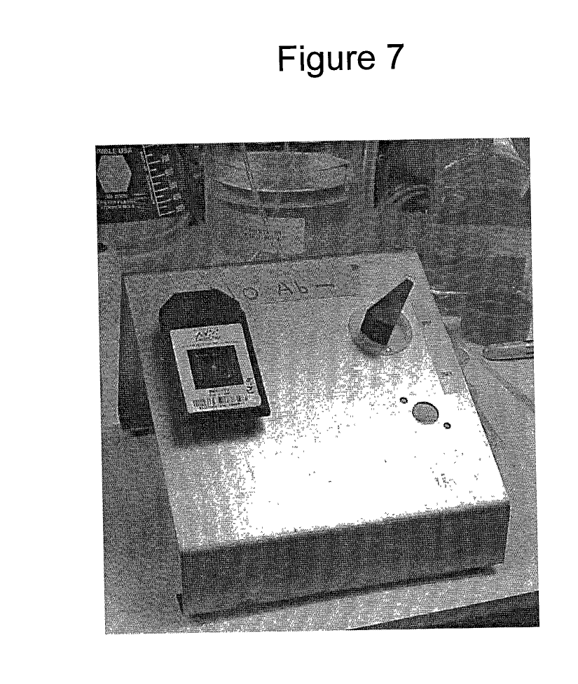

Automation. Custom automation was developed for the laboratory in which several separate “islands” or stations were configured for parts of the sample preparation and assay. For sample preparation and amplification, each station was centered around a Packard Multiprobe Robot. All preparation was done in 96 well plate format and plates were transferred from station to station by hand. For the assay itself, several GeneChip® systems including Hybridization Oven 320/640's, FS 400 Fluidic Stations, and GeneArray Scanners (Affymetrix Inc., Santa Clara Calif.) were used. Several modifications and improvements were made to the GeneChip(R) system. A scanner autoloader for arrays, a faster array wash station, and a linked laboratory tracking and data management system were developed to improve efficiency, and to reduce failure analysis time, array handling time and quantity of reagents required ultimately reducing total costs. The scanner autoloader is a refrigerated unit containing a carousel of 8 racks of 8 arrays (FIG. 6). A robotic arm lifts the array from the carousel and drops it into the scanner while the associated software signals the scan to begin. Once the scan is complete the arm retrieves the scanned array and replaces it in the rack before picking up the next array. All scan information is linked by a barcode placed on the array cartridge and read by the autoloader. A faster wash station prototype (FIG. 7) using vacuum to draw wash solution into the array cartridge and from the cartridge after a short incubation period enabled 12 to 20 arrays to be processed in the same time as 4 arrays processed on the FS 400 fluidics station. Additionally, a special robotics fixture was developed to allow a Multiprobe Robot station to automatically load samples into 24 array cartridges prior to the hybridization step.

The laboratory and data management database, HTS 2000, built for the project was a two-tiered, distributed client/server application developed in MS Visual Basic 6.0 and Oracle8i using ActiveX Data Objects (ADO). With a MS Outlook look and feel, the modular design of the interface mirrors the complex process of high-throughput screening and SNP discovery, from sequence and primer selection to documenting primer testing gel results and the pooling of amplicons for purification, quantification, fragmentation and labeling. Every step of the process from sample preparation to data analysis was tracked and linked by barcode.

Analysis Software. Once an array was scanned a grid was aligned to assign an x,y coordinate to the signal intensity generated at each feature so that subsequent analysis could be carried out. In early expression applications there was little need to automate grid alignment since few people were carrying out many scans in a single day. For SNP discovery applications as well as genotyping many more samples are required therefore there is a need for an automated batch grid alignment tool. In an effort to improve throughput and efficiency a prototype was developed to automatically perform this alignment in batches.

Data Analysis. A new analysis method, ABACUS, Adaptive Background Genotype Calling Scheme, was developed to improve reproducibility and accuracy especially of the heterozygote calls and has been described in detail elsewhere (Cutler et al. 2001). Automated SNP calling and assignment of a confidence score eliminates the need for each call to be individually reviewed and evaluated thus significantly improving consistency, accuracy and throughput while reducing analysis time and cost.

### Results

Early in our study most collaborators produced samples from cDNA by amplifying short fragments less than 1 KB, or amplifying short sequence tag sites, on average less than 200 bps. Those 50-6000 short amplicons were pooled for each hybridization. Precisely measuring and pooling equimolar amounts of large numbers of amplicons was not a trivial undertaking and even the most careful had difficulty carrying this out with enough accuracy to prevent an adverse effect on data quality. In the presence of high and low concentrations of amplicons pooled together and hybridized to one array, it is very difficult to distinguish low abundance signal from background and noise. For instance, since a heterozygote variant sample splits the hybridization intensity between two probes, a sample that is inaccurately quantitated such that concentration is low will generate signal that is not significantly higher than background making accurate base calling impossible. In addition, not all collaborators could afford the time and expense of electrophoresing 50-6000 amplicons for each sample prior to pooling. Consequently, samples were not all quality controlled which resulted in the hybridization of incomplete samples. Missing amplicons were most often the result of inaccurate quantitation and pooling, failed PCR caused by low abundance transcript used in the production of cDNA, inefficient annealing due to the presence of SNPs within a priming region or simply poor quality sample DNA. This resulted in missing data for some fragments for some samples, leading to a loss of power in the analysis.

It was found that PCR failure correlates with the level of abundance of mRNA used for the production of cDNA. Of 281 transcripts detected at less than 5 copies per cell, only 31% of them were amplifiable in more than 35 of 40 samples compared to 81% for the transcripts present at greater than 10 copies per cell (Table 1). In early studies in which collaborators used cDNA as PCR template and amplified short fragments, 8-24% of the amplicons were missing from the samples provided to us for screening.

| Confidence | N | % Confirmed |
|---|---|---|
| High | 67 | 94 |
| Medium | 106 | 82 |
| Low | 86 | 66 |
| Total | 259 | 81 |

As the rough draft of the Human Genome neared completion, the availability of additional sequence information was employed to use genomic DNA and long PCR for sample generation. Long PCR sample preparation offers a number of advantages including reducing the required number of primers which subsequently reduces reagent costs and PCR related handling steps. With this approach there are fewer amplicons to quantitate and pool which leads to more consistent signal intensities across the arrays resulting in better data quality. Using genomic DNA and long PCR an average of 5 amplicons with an average length of 6 kb were pooled per sample. This is a tenfold or greater reduction in the number of PCR reactions, gels to run, and amplicons to quantitate and pool. In these later studies in which long PCR of genomic DNA was used as template the PCR amplification success rose to greater than 94%.

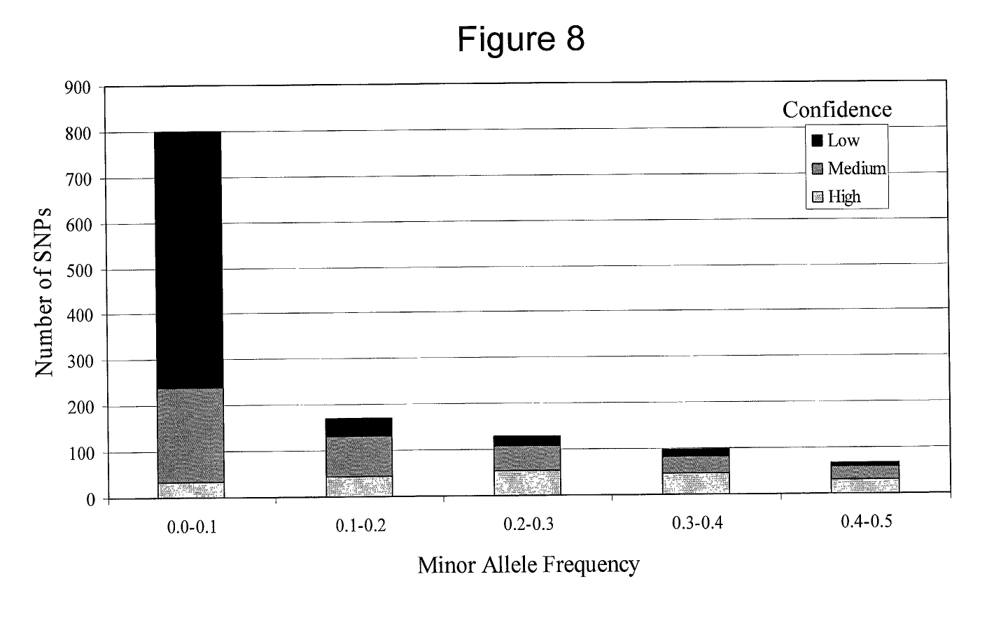

SNP discovery analysis was first performed using an adaptation of the algorithm of Chee M, Yang R, Hubbell E, Berno A, Huang X C, Stem D, Winkler J, Lockhart D J, Morris M S, Fodor S P A. 1996. Accessing genetic information with high-density DNA arrays. Science 274: 610-614. Modifications were made to compensate for using lower signal intensities generated by smaller featured arrays and to perform heterozygote base-calling. The modified analysis method generated candidate SNPs that were independently evaluated by two trained analysts. In an effort to confirm and validate the results obtained by this method we compared our results to the results of single pass sequencing of 328 fragments that had been called with high, moderate or low confidence. Single pass sequencing was performed for each fragment from 2 samples, the reference homozygote case, and the homozygote or heterozygote variant allele case. 81% of the SNPs identified using the modified algorithm of Chee et al., were identical. The most difficult SNPs to confirm were the rare alleles, the largest class of SNPs identified. In this class we were able to confirm only 66% of the SNPs (FIG. 8). Due to the amount of manual analysis time required and poor confirmation performance, it became clear that improvements in throughput and SNP calling accuracy necessitated the development of a new automated analysis method.

One of skill in the art would appreciate that any statistical algorithm must be evaluated using actual genotyping data to select the appropriate algorithm and to develop various parameters for algorithms. A new algorithm, Abacus™, was developed and implemented (Cutler et al. 2001) using genotyping data. Abacus™ automatically performs base-calling, generates a quality score and identifies SNPs using a probability model approach. Four models are considered for the homozygote case. If the sample is a homozygote G, it is assumed that the features representing the other 3 possible nucleotides for this position on the forward strand (C, T, A) are independent and identically distributed and that the intensity information for G will have a different mean and variance. For the homozygote case the three other possible calls are treated in the same way. For the heterozygote case, the data is evaluated with the four homozygote models above plus 6 heterozygote models, G-C, G-T, G-A, A-T, A-C, C-T (Cutler et al., 2001). The likelihood of each model for each base call is calculated independently for both strands and is combined to determine how well the model fits and if it fits sufficiently better than any of the other models. A call is made if one model fits the data significantly better than the other models, the same model must fit both the sense and antisense position and a position which doesn't significantly fit one model better than any other is called N. Additional rules in the analysis software attempt to identify PCR failures that can result in incorrect base calls. Threshold values for these rules can be set by the user. The default settings require that greater than 50% of the bases in the amplicon are callable, that is at least 10/20 surrounding bases must be callable. Also a site must be unambiguously callable, no N's, in greater than 50% of the samples queried. Of course the site does not have to be the same base call in those samples. Base-calling is completely automated which removes analyst bias and greatly reduces analysis time. A confidence score is produced for each base-called thereby providing a means of evaluating the relative risk of including specific SNPs in subsequent studies. The confidence score is the difference between the log (base 10) of the likelihood of the best fit model to the second best fit model. Two types of validation studies were carried out to evaluate our improved process, base calling or genotyping accuracy and SNP calling accuracy. To evaluate base calling accuracy a validation study was carried out comparing array based resequencing with data obtained by 4-8× for 1938 basepairs. 99.998% (1935/1938 basepairs) were called identically with an Abacus confidence score of 1:100,000, high confidence. To validate the SNPs discovered by resequencing, a subset of 117 was selected and 100% of them have been validated by standard sequencing methods.

Automation of sample preparation allowed a reduction in reagent volumes and reduced reagent costs by 33%. Automated array handling and analysis doubled the throughput possible. Ultimately, two skilled research assistants could routinely prepare sample, hybridize and analyze 40 arrays per day. Over the course of the two-year program all or part of 25,051 human genes (8.3 Mb) including some promoter regions were screened in 40 unrelated individuals of 3 different ethnic origins, producing a total of more than 15,000 SNPs which have been deposited in dbSNP (http://www.ncbi.nlm.nih.gov/SNP).

### Conclusion

The scope of the invention should not be limited with reference to the above description, but should instead be determined with reference to the appended claims, along with the full scope of equivalents to which such claims are entitled.

All cited references, including patent and non-patent literature, are incorporated herein by reference in their entireties for all purposes.

What is claimed is:

1. A system for high throughput detection of genotypes comprising
a sample preparation automation system;
a sample tracking system;
an automated high density probe array loader;
a computer system for managing hybridization data and for analyzing hybridization data to make genotype calls.

2. The system of claim 1 wherein the sample preparation automation system is a robotic device for handling multwell plates.

3. The system of claim 1 wherein the sample tracking system is a bar code system.

4. The system of claim 1 wherein computer system comprises a processor; and a memory being coupled with the processor, the memory storing a plurality of machine instructions that cause the processor to perform the method step of analyzing the hybridization to determine the genotype, wherein the analyzing comprises calling a genotype by calculating the likelihood of a set of models for the hybridization and the base is called based upon the likelihood of the models; wherein the distribution of hybridization intensities are assumed to be Gaussein and forward and reverse strand are treated as independent replicates.

5. The method of claim 4 wherein the models are five homozygote Models (Null, A, C, G, T) for a haploid and and 11 models (Null, A, C, G, T, A-C, A-G, A-T, C-G, C-T, G-T) for a diploid.

6. The method of claim 5 wherein likelihood of a model is calculated independently for both the forward and reverse strands and is combined for the overall likelihood of the model.

7. The method of claim 6 wherein a genotype is called if one model fits the hybridization data better than all other models.

8. A method for determining the genotype of a polymorphism comprising:
preparing a nucleic acid sample;
determining the hybridization of the nucleic acid sample with a high density oligonucleotide probe array; wherein the high density oligonucleotide probe array having probes interrogating the polymorphism; and
analyzing the hybridization to determine the genotype, wherein the analyzing comprises calling a genotype by calculating the likelihood of a set of models for the hybridization and the base is called based upon the likelihood of the models.

9. The method of claim 8 wherein the models are five homozygote Models (Null, A, C, G, T) for a haploid and and 11 models (Null, A, C, G, T, A-C, A-G, A-T, C-G, C-T, G-T) for a diploid.

10. The method of claim 9 wherein likelihood of a model is calculated independently for both the forward and reverse strands and is combined for the overall likelihood of the model.

11. The method of claim 10 wherein a genotype is called if one model fits the hybridization data better than all other models.

12. The method of claim 11 wherein the likelihood of a set of hybridization intensity as measured by pixel intensities is:
$$ln ( L ) = - \frac{1}{2} \sum {N}_{r} [ ln ( {\hat{σ}}_{x}^{2} ) + ( {V}_{x} + {M}_{x}^{2} - 2 {\hat{μ}}_{x} {M}_{x} + {\hat{μ}}_{x}^{2} ) / {\hat{σ}}_{x}^{2} + ln ( 2 π ) ] .$$
wherein Nₓ is the number of pixels observed in feature x; Vₓ is the observed variance for feature x, Mₓ is the observed mean for feature x, μₓ is the estimated mean for feature x under a model, and σ²ₓ is the estimated variance for feature x, and wherein the sum is taken over all features x, where x is either A, C, G, or T, on the forward and reverse strands.

13. The method of claim 12 wherein the mean and variance for a Null Model are estimated according to:
$$\begin{matrix}{\hat{μ}}_{r} ( b ) = \frac{{N}_{r} ( A ) {M}_{r} ( A ) + {N}_{r} ( C ) {M}_{r} ( C ) + {N}_{r} ( G ) {M}_{r} ( G ) + {N}_{r} ( T ) {M}_{r} ( T )}{{N}_{r} ( A ) + {N}_{r} ( C ) + {N}_{r} ( G ) + {N}_{r} ( T )} \\ {\hat{μ}}_{f} ( b ) = \frac{{N}_{f} ( A ) {M}_{f} ( A ) + {N}_{f} ( C ) {M}_{f} ( C ) + {N}_{f} ( G ) {M}_{f} ( G ) + {N}_{f} ( T ) {M}_{f} ( T )}{{N}_{f} ( A ) + {N}_{f} ( C ) + {N}_{f} ( G ) + {N}_{f} ( T )} \\ {\hat{σ}}_{f}^{2} ( b ) = \frac{\begin{matrix}{N}_{f} ( A ) ( {V}_{f} ( A ) + {M}_{f}^{2} ( A ) ) + {N}_{f} ( C ) ( {V}_{f} ( C ) + {M}_{f}^{2} ( C ) ) + \\ {N}_{f} ( G ) ( {V}_{f} ( G ) + {M}_{f}^{2} ( G ) ) + {N}_{f} ( T ) ( {V}_{f} ( T ) + {M}_{f}^{2} ( T ) )\end{matrix}}{{N}_{f} ( A ) + {N}_{f} ( C ) + {N}_{f} ( G ) + {N}_{f} ( T )} - {\hat{μ}}_{f}^{2} ( b ) \\ {\hat{σ}}_{r}^{2} ( b ) = \frac{\begin{matrix}{N}_{r} ( A ) ( {V}_{r} ( A ) + {M}_{r}^{2} ( A ) ) + {N}_{r} ( C ) ( {V}_{r} ( C ) + {M}_{r}^{2} ( C ) ) + \\ {N}_{r} ( G ) ( {V}_{r} ( G ) + {M}_{r}^{2} ( G ) ) + {N}_{r} ( T ) ( {V}_{r} ( T ) + {M}_{r}^{2} ( T ) )\end{matrix}}{{N}_{r} ( A ) + {N}_{r} ( C ) + {N}_{r} ( G ) + {N}_{r} ( T )} - {\hat{μ}}_{r}^{2} ( b )\end{matrix}$$

14. The method of claim 10 wherein the mean and variance for a hymozygous model are estimated according to:
$$\begin{matrix}{\hat{μ}}_{f} ( b ) = \frac{{N}_{f} ( C ) {M}_{f} ( C ) + {N}_{f} ( G ) {M}_{f} ( G ) + {N}_{f} ( T ) {M}_{f} ( T )}{{N}_{f} ( C ) + {N}_{f} ( G ) + {N}_{f} ( T )} \\ {\hat{σ}}_{f}^{2} ( b ) = \frac{{N}_{f} ( C ) {ω}_{f} ( C ) + {N}_{f} ( G ) {ω}_{f} ( G ) + {N}_{f} ( T ) {ω}_{f} ( T )}{{N}_{f} ( C ) + {N}_{f} ( G ) + {N}_{f} ( T )} . \\ {ω}_{f} ( x ) = {V}_{f} ( x ) + {M}_{f}^{2} ( x ) - 2 {M}_{f} ( x ) {\hat{μ}}_{f} ( b ) + {\hat{μ}}_{f} ( b ) + {\hat{μ}}_{f}^{2} ( b ) \\ {\hat{μ}}_{f} ( A ) = {M}_{f} ( A ) , \\ {\hat{σ}}_{f}^{2} ( A ) = {V}_{f} ( A ) .\end{matrix}$$
